(19) United States
(12) Patent Application Publication
(10) Pub. No.: US 2009/0311323 A1
(43) Pub. Date: Dec. 17, 2009

(54) Autologous somatic cells from peripheral blood and uses thereof

(76) Inventors: Joaquin Cortiella, Galveston, TX (US); Joan E. Nichols, Galveston, TX (US); Jean A. Niles, Galveston, TX (US); Eric Lee, Houston, TX (US); Donald Prough, Galveston, TX (US)

Correspondence Address: Benjamin Aaron Adler;Adler & Associates, 8011 Candle Lane, Houston, TX, 77071, US

(21) Appl. No.: 11/919,527

(22) PCT Filed: Apr. 28, 2006
(86) PCT No.: PCT/US06/16369

§ 371 Date: Feb. 13, 2009

Related U.S. Application Data
(60) Provisional application No. 60/675,947, filed on Apr. 28, 2005.

(51) Int. Cl. A61K 35/12; C12N 5/08; A61K 38/20; A61K 9/14

(52) U.S. Cl. 424/484; 435/372; 424/93.7; 424/85.2

(57) ABSTRACT

The present invention is directed to developing treatment for spinal cord injury, traumatic brain injury and neural disease using autologous somatic stem cells isolated from peripheral blood. The method identified in the present invention will generate functional neural cells/tissues in order to replace the diseased or damaged neural cells/tissues. In doing so, the cells will not only reverse the motor as well as cognitive dysfunction but will also stabilize the injury site, reduce inflammation and scaring, and halt progressive loss of functional tissue. Further, this method also holds a great promise since it is non-invasive, autologous and can be used acutely.

### CROSS REFERENCE TO RELATED APPLICATION

This non-provisional application claims benefit of provisional application U.S. Ser. No. 60/675,947 filed on Apr. 28, 2005, now abandoned.

### BACKGROUND OF THE INVENTION

1. Field of the Invention

The present invention relates generally to the field of treatment for neurodegenerative diseases. More specifically, the present invention relates to autologous somatic stem cell based treatment for spinal cord injury, traumatic brain injury and neural disease.

2. Description of the Related Art

Traumatic brain injury (TBI) is responsible for numerous deaths and hospitalizations throughout the world. In traumatic brain injury, cognitive and motor dysfunctions are often seen coupled with a degenerative process characterized by moderate to extensive tissue loss. Recent advances in stem cell biology have generated interest in using stem cells as a treatment modality for traumatic brain injury. Mesenchymal, neural and embryonic stem cells have been tried as treatments for traumatic brain injury often with varying results. A stem cell based therapy has the advantage of being non-invasive, autologous and able to be used acutely. Additionally, this treatment is capable of reversing the motor as well as cognitive dysfunction, stabilizing the injury site, reducing inflammation and scaring and halting the progressive loss of functional tissue.

Currently there is no definitive therapy for reversing brain or spinal cord injury. The process of tissue engineering involves the isolation and growth of a patient's autologous cells on biodegradable and non-toxic carrier matrix to produce a polymer/cell construct followed by the delivery of the construct or the engineered tissues back into the recipient. Thus, tissue engineering has shown great promise for the generation of a variety of tissues for which organ donation shortages currently exists, including bone, cartilage, liver and pancreas. However, there has been little investigation of the engineering of neural tissue, with only a few reports focusing on the use of embryonic stem cells as a potential source of stem cells for the use as a potential treatment.

Hence, the prior art is deficient in methods of using embryonic stem cells as a source of stem cells for engineering neural tissue constructs that can be used to reverse brain or spinal cord injury. The present invention fulfills this long-standing need and desire in the art.

### SUMMARY OF THE INVENTION

The present invention involves the development of a treatment for spinal cord injury, traumatic brain injury and neural disease using autologous somatic neural progenitor cells isolated from peripheral blood. The goal of this therapy is to reverse the damage caused by acute or chronic changes in brain or spinal cord (central nervous system) due to disease or traumatic injury.

In one embodiment of the present invention, there is a method of producing neural cells from human peripheral blood-derived neural progenitor cells in vitro. This method comprises collecting human peripheral blood. Neural progenitor cells are isolated from the peripheral blood and cultured in presence of growth factors followed by serum starvation. This induces differentiation of the neural progenitor cells to cells expressing markers associated with neural lineage, thereby producing the neural cells from human peripheral blood-derived neural progenitor cells.

In another embodiment of the present invention, there is provided a composition to treat brain and spinal cord injury. This composition comprises human neural progenitor cells and a bio-acceptable carrier.

In yet another embodiment of the present invention, there is provided a method of treating traumatic brain and spinal cord injury in an individual. This method comprises delivering the above-mentioned composition to the site of injury, thereby treating the traumatic brain and spinal cord injury in the individual.

In still yet another embodiment of the present invention, there is provided a composition to treat brain and spinal cord injury in an individual. This composition comprises human neural progenitor cells and Pluronic F-127 (30%).

In another embodiment of the present invention, there is a method of treating traumatic brain and spinal cord injury in an individual. This method comprises delivering the above-mentioned specific composition to the site of injury, thereby treating brain and spinal cord injury in the individual.

### BRIEF DESCRIPTION OF THE DRAWINGS

FIGS. 1A-D shows the characterization of peripheral stem cells using confocal images. These images show CD34+ peripheral stem cells. FIG. 1A is the isotype staining control, FIG. 1B shows expression of CD34 on the cells, FIG. 1C shows how the cells look like after 12-24 hour transformation and Figure ID shows the expression profile of the surface antigen on the cells.

FIGS. 2A-D show confocal images of cells isolated using the protocol described in the present invention and treated to induce neurogenesis. The cellular development in differentiating cells was characterized using markers specific for nestin (FIG. 2A), neuron-specific nuclear protein (FIG. 2B), neuron-specific tubulin-III (FIG. 2C) and neuron-specific enolase (FIG. 2D). The red arrows indicate the nuclear specific staining of neuron-specific nuclear protein.

FIGS. 3A-B show confocal images at a higher magnification of neural markers in cells prior to being used in the rat-brain engraftment studies. Markers specific for Nestin (FIG. 3A) and Neuron-specific tubulin-III (FIG. 3B) were observed in these cells.

FIGS. 4A-C show confocal images at a higher magnification (630×) of Neuron-specific tubulin III in cells (nuclei counterstained with DAPI) prior to being used in the rat-brain engraftment studies.

FIGS. 5A-B show confocal images at a higher magnification (630×) of Neuron-specific enolase in cells (nuclei counterstained with DAPI) prior to being used in the rat-brain engraftment studies.

FIG. 6 show cells labeled with carboxyfluorescein diacetate, succinimidyl ester (CFSE) prior to implantation into the lateral ventricle of the brain. Staining with a second anti-human specific antibody tagged with a red fluorochrome showed that the engrafted cells were actually of human origin and not scattered rat cells that might have taken up fluorescent debris.

FIGS. 7A-C show confocal images of the cells isolated using the protocol described in the present invention and carboxyfluorescein diacetate, succinimidyl ester labeled and injected into the lateral ventricle of a male rat brain after injury.

FIGS. 8A-B show the results of Morris Water Maze Testing showing comparison of 6 different test situations such as sham, Pluronic F127/stem cell constructs, PBS/Stem cells, Pluronic alone, moderate trauma and peripheral blood mononuclear neural progenitor cells (PBL)/Pluronic F127. All test scenarios contained an N=8. The data suggested that Pluronic with Stem cells, Pluronic F127 were similar to sham suggesting a reversal or marked improvement of a cognitive deficit when compared to the other groups.

FIG. 9 shows a picture of rat brain with different areas.

FIG. 10 shows confocal images of injured brain with Nestin positive cells in hippocampus area (three months after the injury).

FIGS. 11A-C show confocal images of tyrosine hydroxylase expressing cells in the hippocampus and lateral hippocampus area of an injured brain (3 months after injury).

FIGS. 12A-D show higher power of confocal images of engrafted cells within the injured brain (3 months after injury).

FIGS. 13A-H show the change in morphology of the cells to that of glial fibrulary acidic protein (GFAP) expressing astrocyte-like cells. GFAP is an astrocyte marker protein. There was no production of neural markers in CFSE labeled cells. FIG. 13A shows positive staining for CFSE+, FIG. 13B shows no staining for TH, FIG. 13C shows positive staining for CFSE, FIG. 13D shows staining for TH, FIG. 13E shows staining for CFSE, FIG. 13F shows staining for nestin, FIG. 13G shows staining for CFSE and FIG. 13H shows staining for glial fibrulary acidic protein.

FIGS. 14A-G show fluorescent staining for markers in rat brain. FIGS. 14A and 14E are controls, FIGS. 14B show expression of nestin, FIG. 14C shows expression of NSNP, FIG. 14D shows expression of TH, FIG. 14F shows expression of CD45 and FIG. 14G shows expression of CXCR4.

### DETAILED DESCRIPTION OF THE INVENTION

An object of the present invention was to provide a population of stem cells that could be used to generate functional neural cells/tissues in order to replace diseased or damaged neural cells/tissues. The system described in the present invention can be used to create new and functional tissues to treat brain and spinal cord injury and degenerative neural diseases. However, a limitation of the procedure identified in the present invention is that the patient/person must be able to tolerate an initial removal of 50 milliliters of peripheral blood after the injury. To overcome this limitation, progenitor cells from appropriately matched live donors can be used to generate new and engineered functional tissues.

The present invention demonstrated that side-by-side in vitro co-culture experiments of the stem cells (CD34+) with selected neuronal cell lines using a transwell system resulted in the expression of neuronal markers. Co-culture with the astroglial line, svgp12, resulted in expression of glial fibrillar acidic protein (GFAP) and tyrosine hydroxylase (TH). Further, co-culture with the astrocyte line, ditnci, resulted in expression of glial fibrillar acidic protein, tyrosine hydroxylase, nestin, type III tubulin (TYIIITUB) and choline acetyltransferase (CHAT). Additionally, co-culture with neuroblastoma line, sknfi, resulted in expression of glial fibrillar acidic protein, tyrosine hydroxylase, type III tubulin and choline acetyltransferase and co-culture with dopaminanergic line, sknmc, resulted in expression of type III tubulin. However, co-culture of cells with bone marrow stromal cell line, HS-5 did not result in the expression of any neuronal markers listed above.

The present invention also evaluated the ability of human peripheral blood stem cells to attenuate cognitive deficits seen after traumatic brain injury. Isolated human peripheral stem cells were treated with a growth factor mixture twenty-four hours prior to implantation. This treatment resulted in the expression of the neuronal marker, nestin. All cell populations were loaded with carboxy-fluorescein diacetate, succinimidyl ester (CFSE), a membrane impermeant dye. Non-immunosuppressed male Sprague-Dawley rats were anesthetized and subjected to controlled brain injury using a fluid percussion model (n=18) or sham (no) injury (n=8). At 24 hours post-injury, all rats were anesthetized again and randomized to receive steriotactic implantation of the hydrogel carrier (Pluronic F-127) (n=8), treated stem cells in PF-127 (n=8), untreated stem cells in PF-127 (n=8) or treated stem cells in saline (n=8). Implantation of the treated stem cell population in PF-127 into the ventricle resulted in a dramatic improvement in cognition as assessed by Morris Water Maze test as compared to control rats. All animals were sacrificed after 3 months and brains were removed for histological examination. Histological analyses showed that the implanted stem cells survived for 12 weeks. The implanted carboxy-fluorescein diacetate, succinimidyl ester labeled stem cells expressed nestin and tyrosine kinase as evaluated by immunofluoresecence and confocal microscopy.

The present invention is directed to a method of producing neural cells from human peripheral blood-derived mononuclear neural progenitor cells in vitro, comprising: collecting human peripheral blood; isolating the neural progenitor cells from the peripheral blood; culturing the neural progenitor cells in presence of growth factors followed by serum starvation; and inducing differentiation of the neural progenitor cells to cells expressing markers associated with neural lineage, thereby producing the neural cells from the human peripheral blood-derived mononuclear neural progenitor cells.

The peripheral blood may be collected in an acid dextrose vacutainer. Generally, the neural progenitor cells are isolated using size exclusion and cell density counter current centrifugal elutriation in combination with or by size exclusion cell sieving. Specifically, the size exclusion and cell density counter current centrifugal elutriation is Ficoll-Hypaque density gradient cell separation followed by counter current centrifugal elutriation. Additionally, the size exclusion cell sieving is performed using Transwell plates containing 4 μm filter. Moreover, the neural progenitor cells isolated are CD34+ or cells at other stages of development. Further, the growth factors added to induce differentiation are retinoic acid, interleukin-1, tumor necrosis factor-alpha, interleukin-6, fibroblast growth factor or combinations thereof. The isolated neural progenitor cells are cultured for 12-24 hours in the neural induction media followed by 12-24 hours of serum starvation to induce expression of nestin by the differentiating cells. Alternatively, the isolated neural progenitor cells are cultured for 12 hours in the neural induction media followed by 3-8 hours of serum starvation to induce expression of nestin by the differentiating cells. Still further, the cell differentiation is controlled by autologous CD14 monocytes-macrophages or other autologous cell types.

The present invention is also directed to a composition to treat brain and spinal cord injury, comprising human neural progenitor cells and a bio-acceptable carrier. The composition also comprises of growth factors to promote neural development. Representative examples of these growth factors are as discussed above. A bio-acceptable carrier can be a biodegradable and non-toxic carrier such as a matrix. A matrix is a biodegradable and non-toxic carrier that maintains the cells in a three-dimensional orientation during initial placement in the brain. One example of a matrix is a modified hydrogel. This hydrogel may be made of Pluronic-F127 (10-23%). Further, the bio-acceptable carrier may be a buffered salt solution, a cell culture media or a combination of cell culture media and Pluronic-F127 (23%). The human neural progenitor cells used in such a composition may express nestin on cell surface. Such human neural progenitor cells may be derived by inducing differentiation of autologous mononuclear neural progenitor cells isolated from peripheral blood. Additionally, the autologous mononuclear neural progenitor cells isolated are CD34+ or cells at other stages of development.

The present invention is further directed to a method of treating traumatic brain and spinal cord injury in an individual. This method comprises delivering the composition described above to the site of injury, thereby treating the traumatic brain and spinal cord injury in the individual. Such delivery enables the human neural progenitor cells in the composition to engraft in the site of injury.

Additionally, the present invention is also directed to a composition to treat brain and spinal cord injury in an individual comprising human neural progenitor cells and Pluronic F-127 (30%). All other aspects regarding the human neural progenitor cells are the same as described above.

Further, the present invention is directed to a method of treating traumatic brain and spinal cord injury in an individual, comprising delivering the specific composition described above to the site of injury. Such delivery enables the human neural progenitor cells in the composition to engraft in the site of injury.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Isolation and Characterization of Selected Stem Cell Population

The CD34 positive cells were isolated from human peripheral blood as follows. Blood was drawn using acid citrate dextrose as anticoagulant. Peripheral blood “buffy coats” were obtained from Blood Bank or blood was drawn from donors (18-50 years of age) after informed consent. Equal numbers of male and female subjects were used as volunteer donors. Mononuclear cells including neural progenitor cells were isolated after dilution of buffy coats 3:1 or whole blood 1:1 with phosphate buffered saline and layered over density gradient separation medium (Ficoll-paque, Pharmacia).

Adult stem cells were first isolated by overnight adherence of mononuclear neural progenitor cells onto plastic Petri dishes. Non-adherent cells were washed off the plates using warm saline and adherent cells were collected for characterization. The initial adherent population was a mixed monocyte-macrophage/fibroblast/progenitor cell population. Treatment of cells with retinoic acid and 2-Mercaptoethanol induced a morphological change that could be evaluated by flow cytometry or confocal microscopy.

In the later experiments, adult stem cells were isolated by counter current (or counter flow) centrifugal elutriation of peripheral blood cells using a Beckmann J6M elutriator (Beckman Instruments, USA) using a Sanderson chamber. A Masterplex peristaltic pump (Cole Parmer Instruments) was used to fill the system and provide the counter current flow. RPMI 1640 supplemented with 2 mM glutamine, 100 units penicillin G and 100 ug/ml streptomycin and 10% heat inactivated defined fetal calf serum (Hyclone, Utah) was used as elutriation medium. Between 3-6×10⁹ cells were loaded at 3000 RPM and hematopoetic stem cells were isolated using a step-wise reduction of rotor speed until the appropriately sized cell population was isolated. Stem cell enriched populations of lineage-negative (Lin−), CD34+ cells were isolated after removal of both cell culture debris and platelets and lymphocytes. Cells were evaluated for cell size and complexity as each population of cells was sequentially collected after each decrease in rotor speed or increase in fluid flow rate. The cell fractions collected ranged from 8-10 μm range as determined by flow cytometric analysis and Coulter Chanallizer analysis of cells after calibration of forward scatter using 2, 3, 4, 5, 6 and 7, 10 and 20 μm size discrimination beads (Beckmann Coulter). Separated fractions of cells were evaluated by immunophenotyping. This cell separation procedure was shown by flow cytometry to select out, with high purity (100%), mature T, B, natural killer (NK) cells as well as monocytes-macrophages and CD34− cells from the residual Lin-CD34+ cells. These circulating human hematopoetic stem cells were shown to be present at similar numbers in either buffy coat or whole blood preparations.

Fifty milliliters of human peripheral blood was drawn into acid citrate dextrose (ACD) vacutainer tubes. Neural progenitor cells were isolated from whole blood using a Ficoll-Hypaque (Pharmacia) density gradient separation procedure as described by the manufacturer. After washing and resuspension of the cells in DMEM low glucose with 10% FBS, the cells were incubated for 72 hours in the upper chamber of a Transwell plate containing a 4 μm filter (Coming Inc.). After this 72-hour sieving step, cells in the top chamber of the plate were harvested, evaluated for viability using trypan blue and placed in a 175 ml flask, at a concentration of approximately 5×10⁷ cells/ml.

### EXAMPLE 2

### Staining for Analysis of Cell Phenotype

Phenotypes of cells were determined using monoclonal antibodies to identify lymphocyte subsets (BD Pharmingen). Antibodies were conjugated to either FITC, PE or PerCP and corresponding immunoglobulin (IgG) matched isotype control antibodies from each company were used to set baseline values for analysis markers. In all experiments, cultured cells were used as negative controls to set parameters for evaluation of positive levels of cell surface marker expression. After fixation in 2% paraformaldehyde (PAF) cells were stored at 4° C. until analyzed.

### EXAMPLE 3

### Induction of a Neural Lineage

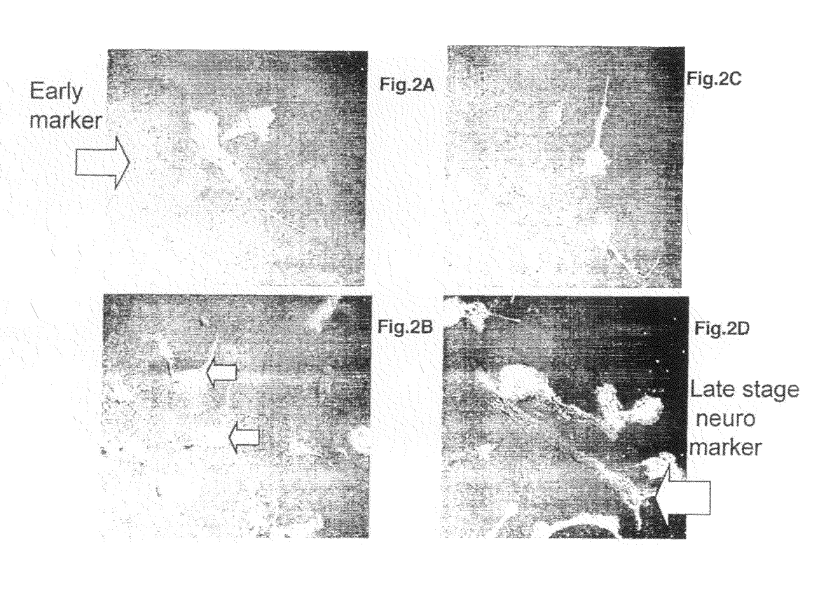

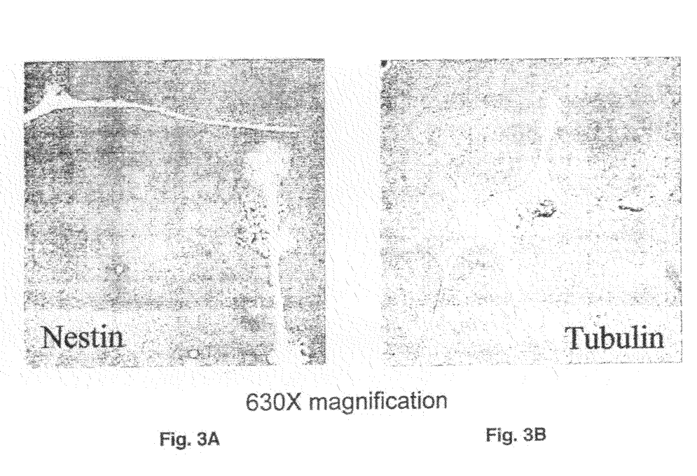

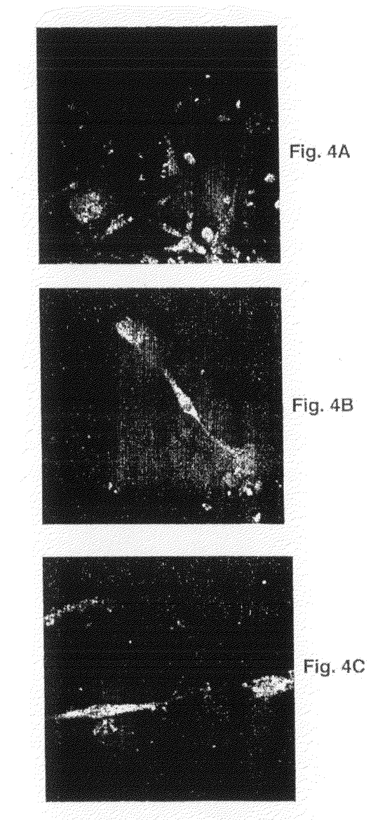

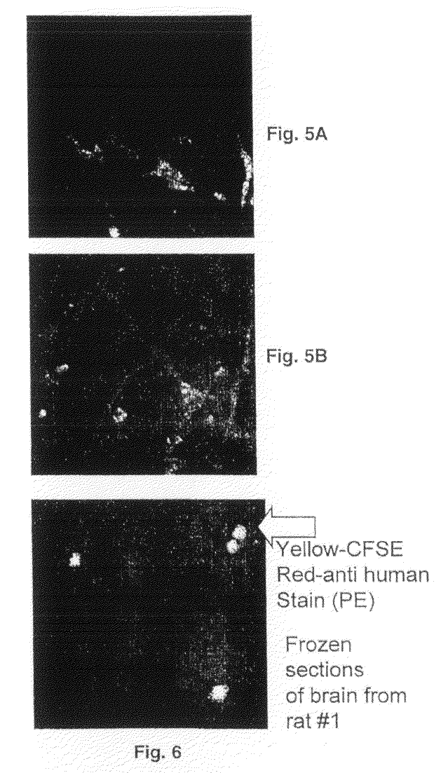

Under certain specific conditions, culture of isolated peripheral blood stem or progenitor cells resulted in the production of cells expressing markers associated with cells of neural lineage. In these experiments, cells were induced to express nestin (FIGS. 2A-3A), neuron-specific nuclear protein (FIG. 2B), neuron-specific tubulin III (FIGS. 2C-3B) and neuron-specific enolase (FIG. 2D) after 7 days of culture and 14 days of culture (FIGS. 4A-C and 5A-B).

Side-by-side in vitro co-culture experiments of these stem cells (CD34+) with selected neuronal cell lines using a transwell system resulted in the expression of neuronal markers. Co-culture with the astroglial line, svgp12, resulted in expression of glial fibrillar acidic protein (and tyrosine hydroxylase. Co-culture with the astrocyte line, ditnci, resulted in expression of glial fibrillar acidic protein, tyrosine hydroxylase, nestin, type III tubulin and choline acetyltransferase. Co-culture with the neuroblastoma line, sknfi, resulted in the expression of glial fibrillar acidic protein, tyrosine hydroxylase, type III tubulin and choline acetyltransferase. Co-culture with the dopaminanergic line, sknmc, resulted in expression of type III tubulin. Co-culture of cells with bone marrow stromal cell line HS-5 did not result in the expression of any of the neuronal markers listed above. Flow cytometric evaluation of the individual neuronal cell lines showed that svgp12 expressed TH (31%) and nestin (3%); ditnci expressed type III tubulin (46%) and nestin (3%); AGAL ( Please confirm that this is right) (7%), sknf1 expressed type Ill tubulin (25%) and tyrosine hydroxylase (8%); sknmc expressed glial fibrillar acidic protein (13%), tyrosine hydroxylase (37%) and nestin (14%).

These cells were cultured in DMEM low glucose (Gibco) with 10% FBS (defined FBS Hyclone) and 1×10⁻³M β-mercaptoethanol (Sigma), 5×10⁻⁷M all-trans-retinoic acid (Sigma). After 24 hours of culture, cells were centrifuged and placed in serum free media (DMEM low glucose) with 5×10⁻⁷M retinoic acid (Sigma). The cells were then cultured and aliquots of cells were removed for analysis of markers specific for neural cell analysis at 12, 24, 48 and 72 hours.

### EXAMPLE 4

Implantation into Rat Brain

To prepare cells for implantation, cells were labeled with carboxyfluroscein diacetate, succinimidyl ester (CMFDA) after 72 hours as follows. Upon passively entering the cell, carboxyfluroscein diacetate, succinimidyl ester is cleaved by cellular esterases and acquires the fluorescence characteristics of fluorescein. Therefore, a decrease in carboxyfluroscein diacetate, succinimidyl ester fluorescence signal will occur if cell division is occurring. Cells were labeled by culturing isolated adult lung cells with carboxyfluroscein diacetate, succinimidyl ester solution (Molecular Probes) a concentration of 2.5 μM in RPMI-1640 for 8 minutes at 37° C. (1×10⁷ cells/ml). After incubation, cells were washed with RPMI-1640 at 4° C. Cells were then cultured in RPMI-1640 (Gibco) with 10% Fetal Calf Serum (FCS) (Hyclone) at 37° C. until polymer/cell constructs were produced as described below.

SLPC/hydrogel composites were created by seeding a 10-23% solution of a reverse thermosetting polymer hydrogel, Pluronic F-127, with 5×10⁷ cells/ml, which were carboxyfluroscein diacetate, succinimidyl ester labeled and then cultured for 4 hours at 37° C. and 5% CO₂. The engraftment potential of these cells was evaluated by implantation of the cells/pluronic F-127 mixture in the right hand ventrical of a 4 month-old male rat. The animal was observed for a period of two weeks for any signs of rejection, sepsis or infection. The animals appeared healthy and were sacrificed after two weeks using the standard guillotin techniques with the brain frozen in −80° C. freezer. Brains were evaluated by a fluorescent microscope for fluorescent staining of the engrafted cells.

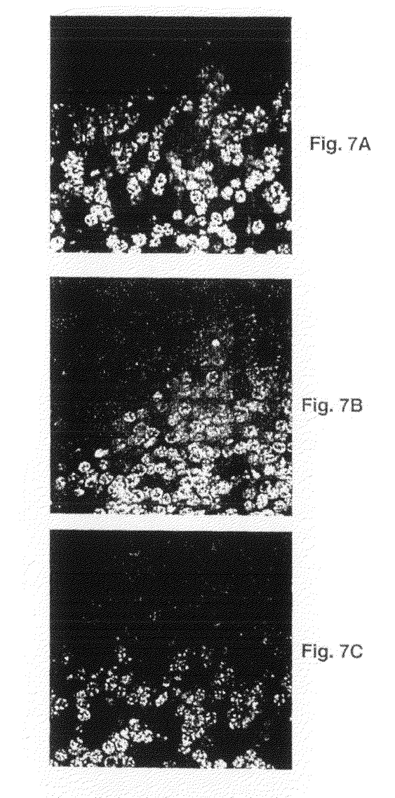

Frozen sections of the whole brain showed that the cells had survived and also showed areas of specific engraftment (FIGS. 7A). Brains were evaluated by using confocal microscopy for fluorescent staining of engrafted cells as well as laser capture of selected cell populations for RNA analysis and cDNA array evaluation. Staining with anti-human antibody showed that the cells were indeed human and only carboxyfluroscein diacetate, succinimidyl ester labeled cells stained positive with anti-human marker.

Confocal images of cells isolated using the above protocol and treated to induce neurogenesis were examined. Markers specific for nestin, neuron-specific nuclear protein, neuron-specific tubulin III and neuron-specific enolase were used to characterize cellular development in differentiating cells. The red arrows in the figure indicate the nuclear specific staining of neuron-specific nuclear protein. FIGS. 2A, 2C show confocal images at a higher magnification of nestin and neuron-specific tubulin III expression respectively, in cells prior to being used in the rat-brain engraftment studies.

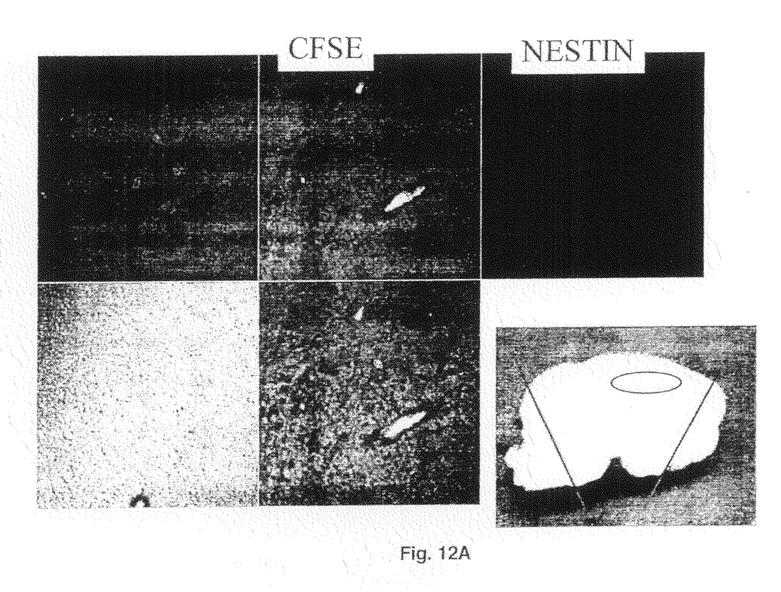

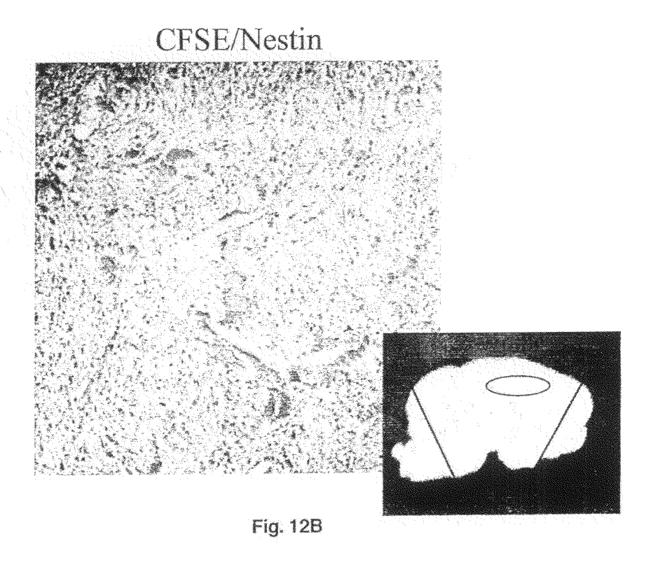

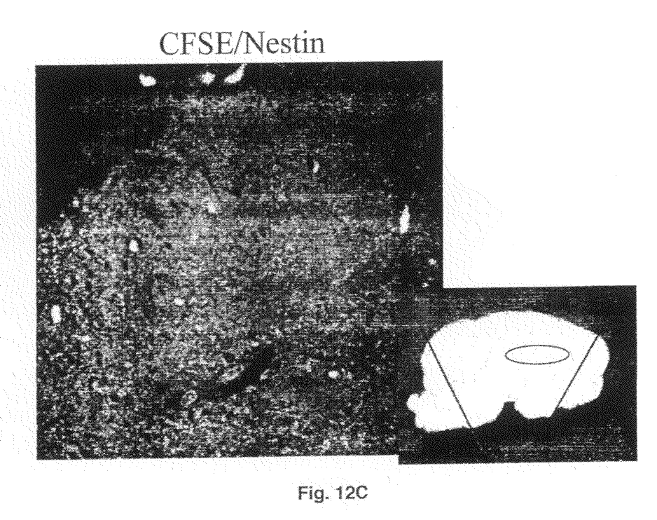

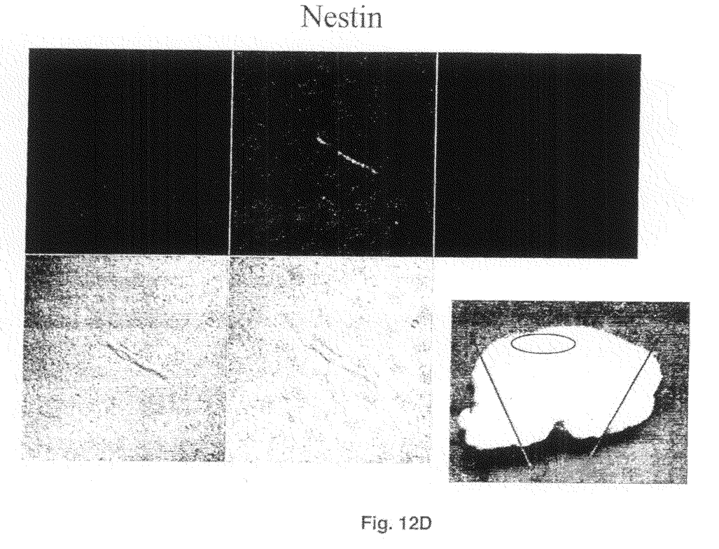

In order to test the functional ability of the neuronal lineage primed cells, these cells were labeled with carboxy-fluorescein diacetate, succinimidyl ester prior to implantation into the lateral ventricle of the brain in a rat injury model. Staining with a second anti-human specific antibody tagged with red fluorochrome in FIG. 12 showed that the engrafted cells were actually of human origin and not scattered rat cells that may have taken up fluorescent debris. Confocal images of cells isolated using the above protocol after carboxy-fluorescein diacetate, succinimidyl ester labeling and implantation into the lateral ventricle of the male rat brain after injury showed isolated fluorescent cells (FIGS. 7B-C).

### EXAMPLE 5

### Fluid Percussion Injury Experiments

Injury-implantation experiments were done on 6 groups of male Sprague-Dawley rats (8 per group). A moderate (2.0:0.1 atm) lateral fluid percussion injury (FPI) was performed as previously described (Mathew, B. P. et al., 1999; McIntosh, T. K. et al., 1989). Briefly, rats were anesthetized with 4% isoflurane in an anesthetic chamber, intubated and mechanically ventilated with 1.5-2.0% isoflurane in O₂/room air (30:70) using a volume ventilator. All rats were placed afterwards in a stereotaxic head holder, after which a midline incision of the skin was performed and skull exposed. With the use of a Michele trephine, a craniotomy was performed lateral to the saggital suture, midway between lambda and bregma sutures, under continuous cooling of the bone with saline. The bone flap was removed, with the dura remaining intact at the site. A modified 20-guage-needle hub was secured in place over the exposed dura with cyanoacrylic adhesive and cemented in place with hygienic dental acrylic. The trauma was administered by means of a trauma device consisting of a Plexiglass cylinder 60 cm long and 4.5 cm in diameter filled with isotonic saline, one end of which was connected to a hollow metal cylinder housing a pressure transducer (Statham PA856-100, Data Instruments, Acton, Mass.) and the other end of which was closed by a Plexiglas piston mounted on O rings. The transducer housing was connected to the rat by a plenum tube cemented to a craniotomy trephined in the skull. To induce traumatic brain injury, a 4.8 kg steel pendulum struck the piston after being dropped from a variable height that determined the intensity of the injury. The pressure pulse was recorded on a storage ocilloscope triggered photoelectrically by the descent of the pendulum.

### EXAMPLE 6

### Morris Water Maze Testing

24 hours after injury, rats were re-anesthetized and control or experimental samples were placed in the lateral ventricle. Rats were allowed to recover and were tested using the Morris water maze learning paradigm after the lateral fluid percussion injury in order to evaluate the behavioral response as described previously (Fujomoto S. T. et al., 2004).

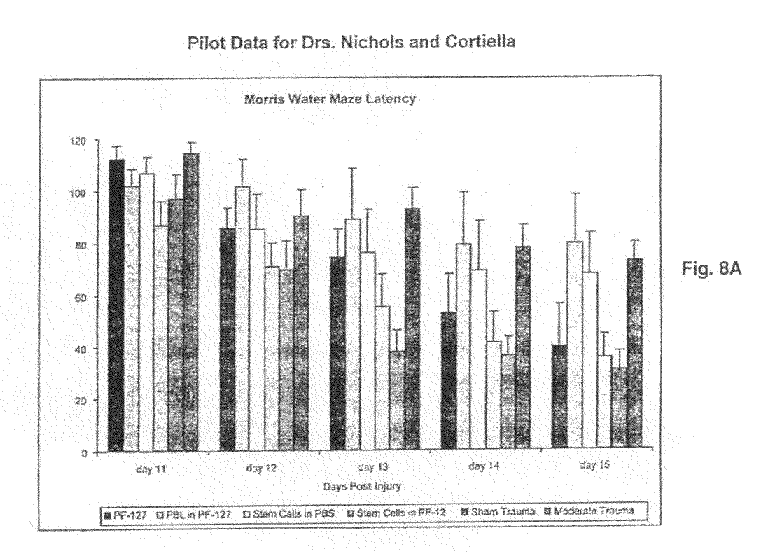

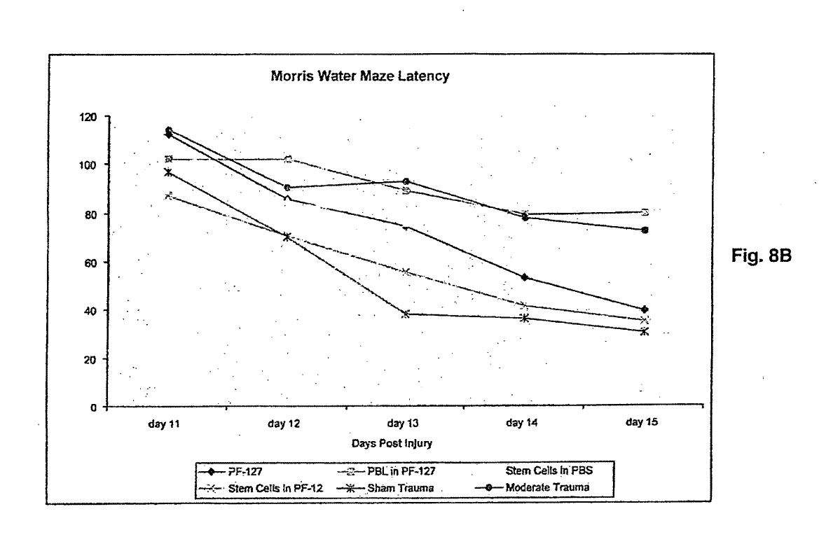

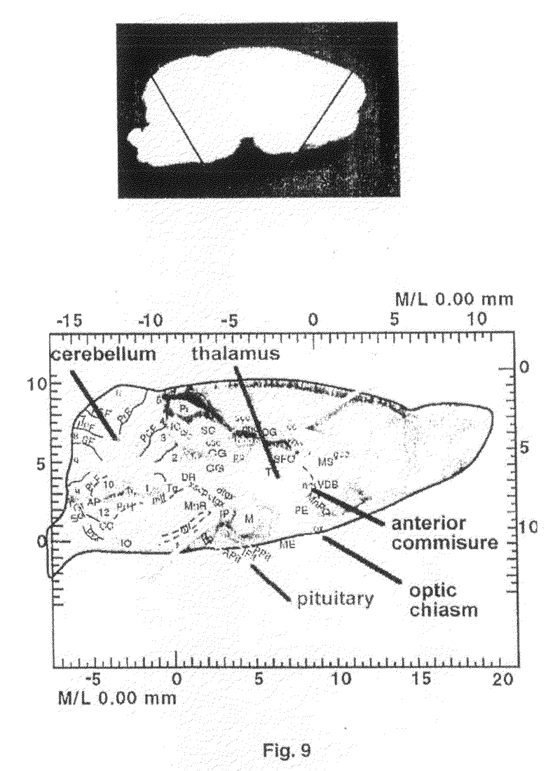

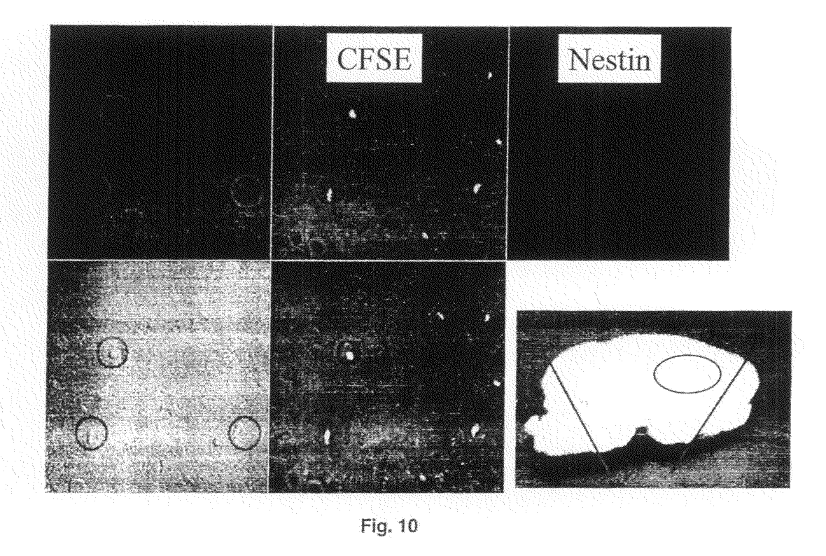

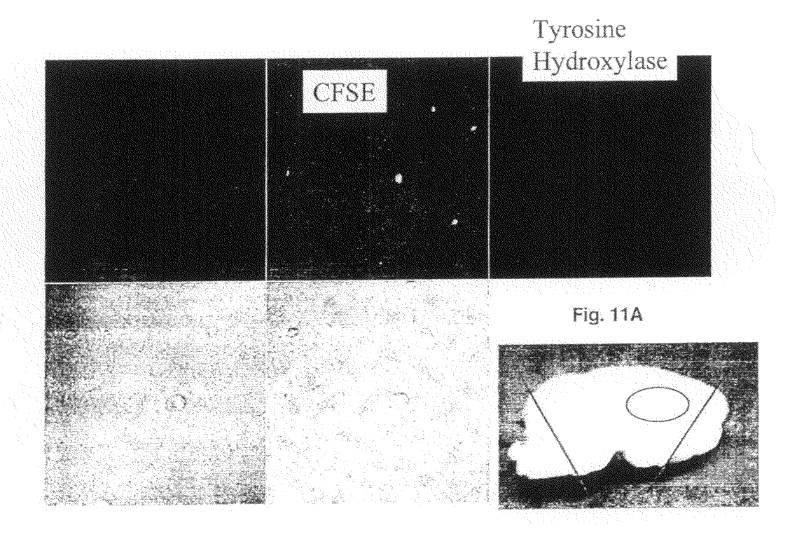

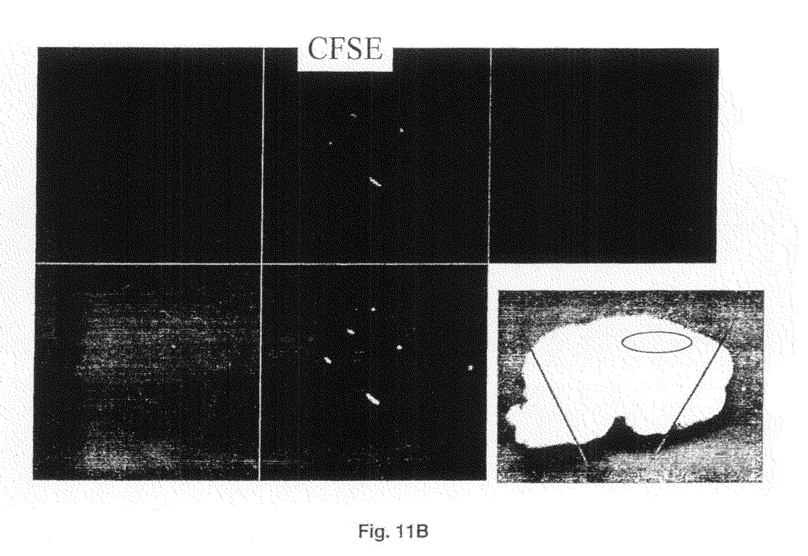

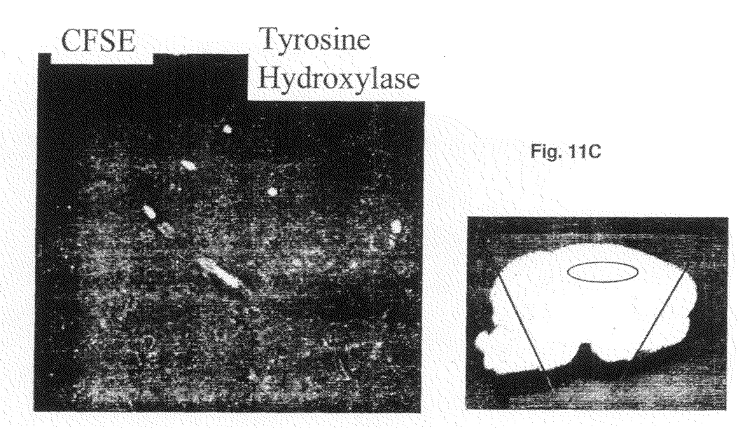

Results of the water maze shown in FIG. 8B (blue line) show that both implantation of PF-127 containing 1×10⁵ neural lineage induced human peripheral blood derived stem or progenitor cells improved the ability of the FPI-treated rats almost to levels of sham treated or uninjured rats. Treatment with PF-127 alone also improved the functionality of the rats in the maze test but not to the levels seen for cell-treated rats.

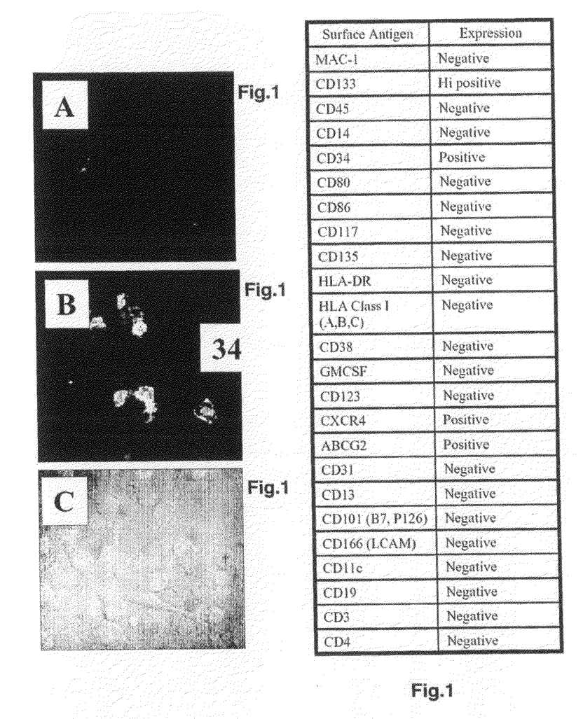

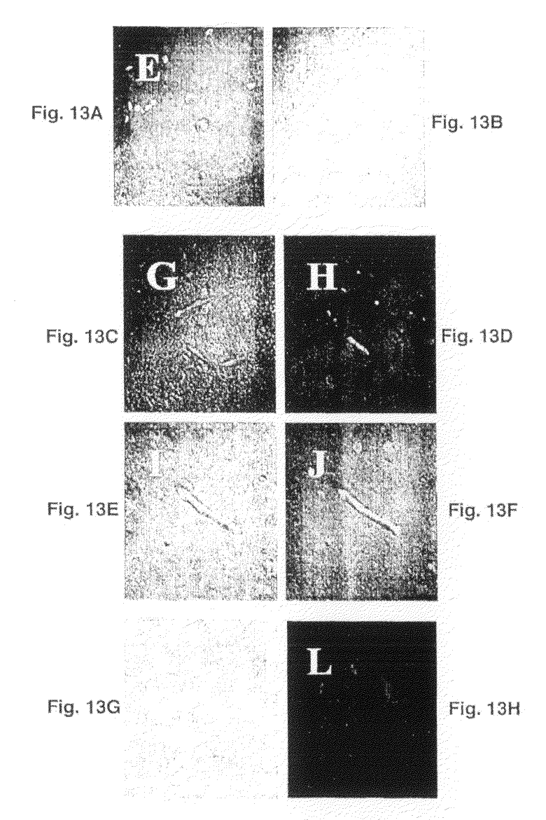

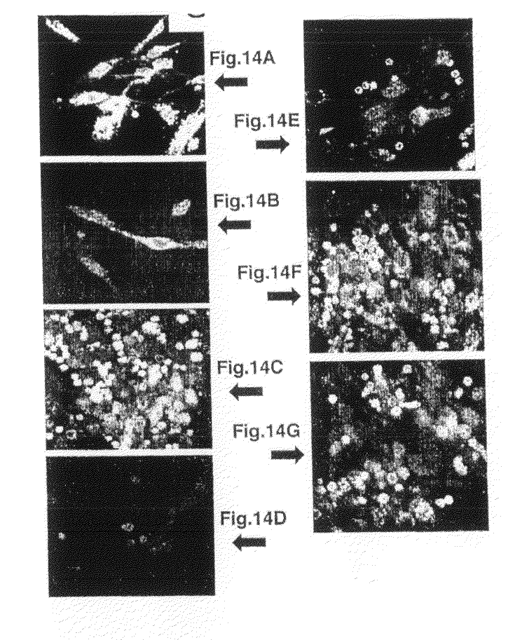

Evaluation of function was done by seeding a 30% solution of a reverse thermosetting polymer hydrogel, Pluronic F-127 with 5×10⁷ cells/ml that were CFSE-labeled cells into traumatic brain injured rat. The rats were then sacrificed and frozen sections of the whole brain examined. These sections of the brain showed that the cells had survived along with the areas of specific engraftment. Paraffin sections (8 um) of the brains of the rats that showed improvement in the animal testing after traumatic brain injury were stained for neural markers (FIGS. 13A-H). No neural markers were seen in the CFSE labeled cells. Additionally, cells were also stained with fluorescent antibodies to determine the expression of nestin (FIG. 14B), NSNP (FIG. 14C), TH (FIG. 1D), CD45 (FIG. 14F) and CXCR4 (FIG. 14G).
The following references were cited herein:
   Fujomoto et al. (2004) Neuroscience and biobehavioral reviews 28: 365-378.

### Mathew, B. P. et al. (1999) J Neurotrauma 16: 1177-1186.

### Mcintosh, T. K. et al. (1989) Neuroscience 28: 233-244.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A method of producing neural cells from human peripheral blood-derived neural progenitor cells in vitro, comprising:
collecting human peripheral blood;
isolating the neural progenitor cells from the peripheral blood;
culturing the neural progenitor cells in presence of growth factors followed by serum starvation; and
inducing differentiation of the neural progenitor cells to cells expressing markers associated with neural lineage, thereby producing the neural cells from the human peripheral blood-derived neural progenitor cells.

2. The method of claim 1, wherein said peripheral blood is collected in acid citrate dextrose vacutainer.

3. The method of claim 1, wherein said neural progenitor cells are isolated using size exclusion and cell density counter current centrifugal elutriation in combination with or by size exclusion cell sieving.

4. The method of claim 3, wherein the size exclusion and cell density counter current centrifugal elutriation is Ficoll-Hypaque density gradient cell separation followed by counter current centrifugal elutriation.

5. The method of claim 3, wherein the size exclusion cell sieving is performed using Transwell plates containing 4 μm filter.

6. The method of claim 1, wherein said neural progenitor cells isolated are CD34+ or cells at other stages of development.

7. The method of claim 1, wherein the growth factors added to promote neural development are retinoic acid, interleukin-1, tumor necrosis factor-alpha, interleukin-6, fibroblast growth factor or combinations thereof.

8. The method of claim 1, wherein said isolated neural progenitor cells are cultured for 12-24 hours in the neural induction media followed by 12-24 hours of serum starvation to induce expression of nestin by the differentiating cells.

9. The method of claim 1, wherein said isolated neural progenitor cells are cultured for 12 hours in the neural induction media followed by 3-8 hours of serum starvation to induce expression of nestin by the differentiating cells.

10. The method of claim 1, wherein said cell differentiation is controlled by autologous CD14 monocytes-macrophages or other autologous cell types.

11. A composition to treat brain and spinal cord injury, comprising a human neural progenitor cells and a bio-acceptable carrier.

12. The composition of claim 11, wherein said composition further comprises growth factors to promote neural development.

13. The composition of claim 12, wherein said growth factors are retinoic acid, interleukin-1, tumor necrosis factor-alpha, interleukin-6, fibroblast growth factor or combinations thereof.

14. The composition of claim 11, wherein said bioacceptable carrier is a matrix.

15. The composition of claim 14, wherein said matrix is a modified hydrogel.

16. The composition of claim 15, wherein said hydrogel is made of Pluronic-F127 (10-23%).

17. The composition of claim 11, wherein said bio-acceptable carrier is a buffered salt solution.

18. The composition of claim 11, wherein said bio-acceptable carrier is cell culture media.

19. The composition of claim 11, wherein said bio-acceptable carrier is a combination of a cell culture media and Pluronic-F127 (23%).

20. The composition of claim 11, wherein said human neural progenitor cells express nestin on cell surface.

21. The composition of claim 11, wherein said human neural progenitor cells are derived by inducing differentiation of autologous mononuclear neural progenitor cells isolated from peripheral blood.

22. The composition of claim 21, wherein said autologous mononuclear neural progenitor cells isolated are CD34+ or cells at other stages of development.

23. A method of treating a traumatic brain and spinal cord injury in an individual, comprising:
delivering the composition of claim 11 to site of injury, thereby treating the traumatic brain and spinal cord injury in the individual.

24. The method of claim 23, wherein said delivery enables the human neural progenitor cells in the composition to engraft in the site of injury.

25. A composition to treat brain and spinal cord injury in an individual, comprising human neural progenitor cells and Pluronic F-127 (30%).

26. The composition of claim 25, wherein said human neural progenitor cells are derived by inducing differentiation of autologous neural progenitor cells isolated from peripheral blood.

27. The composition of claim 26, wherein said neural progenitor cells isolated are CD34+ or cells at other stages of hematopoetic development.

28. The composition of claim 25, wherein said human neural progenitor cells express nestin intracellularly.

29. A method of treating traumatic brain and spinal cord injury in an individual, comprising:
delivering the composition of claim 25 to a site of injury, thereby treating the traumatic brain and spinal cord injury in the individual.

30. The method of claim 29, wherein said delivery enables the human neural progenitor cells in the composition to engraft in the site of injury.
